(19) United States
(12) Patent Application Publication
(10) Pub. No.: US 2021/0378508 A1
(43) Pub. Date: Dec. 9, 2021

(54) Methods and Systems for Determining Wavefronts for Forming Optical Structures in Ophthalmic Lenses

(71) Applicant: Clerio Vision, Inc., Rochester, NY (US)

(72) Inventor: Leonard Zheleznyak, Pittsford, NY (US)

(73) Assignee: Clerio Vision, Inc., Rochester, NY (US)

(21) Appl. No.: 17/339,713

(22) Filed: Jun. 4, 2021

Related U.S. Application Data
(60) Provisional application No. 63/035,294, filed on Jun. 5, 2020.

(51) Int. Cl. A61B 3/10; A61B 3/103; G02C 7/02; G02C 7/04

(52) U.S. Cl. CPC A61B 3/1015; A61B 3/103; G02C 2202/24; G02C 7/041; G02C 7/022

(57) ABSTRACT

Embodiments include methods and systems forming optical structures in an ophthalmic lens for improving a patient's vision by accessing a prescription for the patient; generating a variable wavefront based on the prescription; phase wrapping the first variable wavefront, wherein phase wrapping the first variable wavefront includes collapsing the first variable wavefront to a phase-wrapped wavefront having a predetermined phase height; and generating, based on the phase-wrapped wavefront, energy output parameters for forming an optical structure in the ophthalmic lens using an energy source.

### CROSS-REFERENCE TO RELATED APPLICATIONS

This application claims priority to U.S. 63/035,294, filed on Jun. 5, 2020. The disclosure of which is hereby incorporated by reference in its entirety and for all purpose.

### BACKGROUND

Optical aberrations that degrade visual acuity are common. Optical aberrations are imperfections of the eye that degrade focusing of light onto the retina. Common optical aberrations include lower-order aberrations (e.g., astigmatism, positive defocus (myopia) and negative defocus (hyperopia)) and higher-order aberrations (e.g., spherical aberrations, coma and trefoil).

Existing treatment options for correcting optical aberrations include glasses, contact lenses, and reshaping of the cornea via laser eye surgery. Additionally, an intraocular lens is often implanted in an eye. For example, an intraocular lens can be implanted to replace a native lens removed during cataract surgery.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Embodiments described herein are directed to ophthalmic lenses that include at least one subsurface annular optical structure (e.g., diffractive optical structures and/or non-diffractive optical structures) with enhanced distribution of refractive index values. In many embodiments, the subsurface refractive index variations are formed via focusing femtosecond duration laser pulses onto a targeted sequence of subsurface volumes of an ophthalmic lens. The refractive indexes of the annular optical structure vary radially relative to the optical axis up to an upper limit refractive index (e.g., providing any suitable phase change less than 1.0 wave). The refractive indexes of the annular optical structure are equal to the upper limit refractive index over a range of radii (e.g., at least 0.15 mm length) from the optical axis. In many embodiments, the refractive indexes of the annular optical structure are equal to a lower limit refractive index (e.g., providing a phase change of 0.0 waves) over a range of radii (e.g., at least 0.15 mm length) from the optical axis. The enhanced distribution of refractive index values can be formed using fewer laser pulses in comparison with a corresponding distribution of refractive index values determined via a ratio approach. Additionally, limiting the refractive index values to equal to or less than the upper limit refractive index helps to reduce damage induced by the sequence of laser pulses at a given pulse energy level as compared to forming a corresponding subsurface optical structure(s) using refractive index values that are greater than the upper limit refractive index. The approaches described herein may be useful in forming a subsurface optical structure(s) in any suitable ophthalmic lens (e.g., intraocular lens such as a prosthetic intraocular lens for cataract surgery, a prosthetic anterior chamber lens, a native crystalline lens, or a corneal inlay; a contact lens, a cornea, glasses, and/or a native lens).

In some embodiments, methods, systems, and devices are described for determining parameters for forming an optical structure (e.g., a subsurface optical structure) in an ophthalmic lens for improving vision in a patient. These parameters may be used to control an energy source to appropriately form the desired optical structure. Methods may include accessing a first optical prescription for the patient, wherein the first optical prescription comprises one or more prescription parameters for refracting light directed at a retina of the patient so as to improve vision; generating a first variable wavefront based on the first optical prescription, wherein the first variable wavefront comprises at least one portion that has a phase height greater than 1.0 wave; phase wrapping the first variable wavefront, wherein phase wrapping the first variable wavefront comprises collapsing the first variable wavefront to a first phase-wrapped wavefront having a first predetermined phase height; and generating, based on the first phase-wrapped wavefront, energy output parameters for forming a first subsurface optical structure in an ophthalmic lens using an energy source, wherein the first subsurface optical structure is configured to refract light directed at the retina of the patient so as to improve vision. Systems and devices for implementing these steps are disclosed herein. In some embodiments, the generated energy output parameters may be used to control an energy source, or may be sent to an energy delivery system for controlling an energy source of the energy delivery system.

The one or more prescription parameters may include diopter values of sphere, cylinder, or axis. In some embodiments, the first variable wavefront may include a two-dimensional wavefront. In some embodiments, the energy source comprises a laser. In some embodiments, the ophthalmic lens is an intraocular lens (e.g., any lens within an eye), a contact lens, or a cornea of the patient. In some embodiments, generating the energy output parameters includes applying a calibration function based on a material property of the ophthalmic lens, a gender of the patient, or an age of the patient. In some embodiments, generating the energy output parameters comprises applying a calibration function based on a depth at which the first subsurface optical structure is to be formed in the ophthalmic lens.

In some embodiments, collapsing the first variable wavefront may include identifying a first discrete segment of the first variable wavefront; reducing a phase height of the first discrete segment by a first scalar such that a peak of the first discrete segment is at the first predetermined phase height; identifying a second discrete segment of the first variable wavefront, wherein the second discrete segment is substantially concentric with the first discrete segment; and reducing a phase height of the second discrete segment by a second scalar such that a peak of the second discrete segment is at the first predetermined phase height. In some embodiments, the first subsurface optical structure is configured to improve presbyopia, and wherein the first predetermined phase height is less than 1.0 wave. Alternatively, the first predetermined phase height may be greater than 1.0 wave.

In some embodiments, the first predetermined phase height may be 1.0 wave. The first subsurface optical structure in some of these embodiments may be configured to improve myopia, for example. In some embodiments, the method may include generating a second variable wavefront based on a second optical prescription, wherein the second optical prescription comprises an add power for multifocal vision correction; and phase wrapping the second variable wavefront, wherein phase wrapping the second variable wavefront comprises collapsing the second variable wavefront to a second phase-wrapped wavefront having a second predetermined phase height, wherein the second predetermined phase height is less than 1.0 wave. In some embodiments, the method may further include generating, based on the second phase-wrapped wavefront, energy output parameters for forming a second subsurface optical structure in an optical structure using an energy source, wherein the second subsurface optical structure is configured to diffract light so as to create multiple focal points. In some embodiments, the first subsurface optical structure is configured to improve myopia and the second subsurface optical structure is configured to improve presbyopia, the first subsurface optical structure and the second subsurface optical structure in combination forming a multifocal refractive structure. In some embodiments, the energy output parameters for forming the first subsurface optical structure are further based on the second phase-wrapped wavefront such that the first subsurface optical structure is configured to be a single multifocal subsurface optical structure.

In some embodiments, the energy output parameters specify a plurality of power levels corresponding to a plurality of optical zones on the ophthalmic lens. The method may further include: directing a first energy beam from the energy source at a first optical zone on the ophthalmic lens for a first duration, wherein a power level of the first energy beam is based on a corresponding power level as specified by the energy output parameters; and directing a second energy beam from the energy source at a second optical zone on the ophthalmic lens for a second duration, wherein a power level of the second energy beam is based on a corresponding power level as specified by the energy output parameters. The first energy beam and the second energy beam may alter refractive indexes of the first optical zone and the second optical zone, respectively, and the first subsurface optical structure may include the first optical zone and the second optical zone. The first subsurface optical structure may be formed within an interior of the ophthalmic lens.

The disclosure describes particular devices and systems for implementing various steps of methods such as those discussed briefly above, but it contemplates any suitable devices and systems for implementing the disclosed steps.

### BRIEF DESCRIPTION OF THE DRAWINGS

FIG. 1 is a plan view illustration of an ophthalmic lens that includes subsurface optical structures with enhanced distribution of refractive index variations, in accordance with embodiments.

FIG. 2 is a plan view illustration of a layer of the subsurface optical structures of the ophthalmic lens of FIG. 1.

FIGS. 3A-3B illustrate example wavefronts through a medium for parallel and converging rays of light.

FIGS. 3C-3D illustrate example wavefronts that may simulate aberrations of the eye.

FIG. 3E illustrates a two-dimensional wavefront map and a corresponding first variable wavefront.

FIG. 3F illustrates a first phase-wrapped wavefront corresponding to the first variable wavefront.

FIG. 4 illustrates a second phase-wrapped wavefront having a phase height less than 1.0 wave.

FIG. 5 illustrates a two-dimensional map representation of a phase-wrapped wavefront phase-wrapped at an optical phase height less than 1.0 wave, such as the wavefront in FIG. 4.

FIG. 6 illustrates an example of an optical structure having diffractive properties.

FIG. 7 is a graph illustrating the relative distribution of light between a near-vision focal point and a far-vision focal point as phase height of a wavefront is adjusted between 0 wave and 1.0 wave.

FIG. 8 illustrates a cross section of an ophthalmic lens including a subsurface optical structure having multiple substructures.

FIGS. 9A-9B illustrate example conceptualizations of an ophthalmic lens having a plurality of optical zones.

FIG. 10 illustrates an example method for determining parameters for forming a subsurface optical structure for improving vision in a patient.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

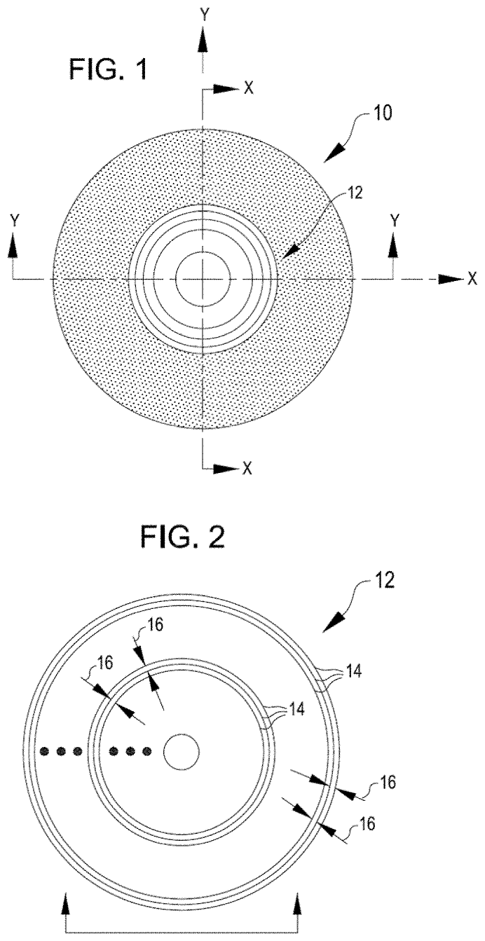

FIG. 1 is a plan view illustration of an ophthalmic lens 10 that includes one or more subsurface optical structures 12 with annular distribution of refractive index variations, in accordance with embodiments. The one or more subsurface structures 12 described herein can be formed in any suitable type of ophthalmic lens including, but not limited to, intra-ocular lenses, contact lenses, corneas, spectacle lenses, and native lenses (e.g., a human native lens). The one or more subsurface optical structures 12 with annular distribution of refractive index variations can be configured to provide a suitable refractive correction for each of many optical aberrations such as astigmatism, myopia, hyperopia, spherical aberrations, coma and trefoil, as well as any suitable combination thereof

FIG. 2 is a plan view illustration of one of the subsurface optical structures 12 of the ophthalmic lens 10. The illustrated subsurface optical structure 12 includes concentric circular sub-structures 14 separated by intervening line spaces or gaps 16. In FIG. 2, the size of the intervening line spaces 16 is shown much larger than in many actual embodiments. For example, example embodiments described herein have an outer diameter of the concentric circular sub-structures 14 of 3.75 mm and intervening line spaces 16 of 0.25 μm, thereby having 1,875 of the concentric circular sub-structures 14 in embodiments where the concentric circular substructures 14 extend to the center of the subsurface optical structure 12. Each of the concentric circular sub-structures 14 can be formed by focusing suitable laser pulses onto contiguous sub-volumes of the ophthalmic lens 10 so as to induce changes in refractive index of the sub-volumes so that each of the sub-volumes has a respective refractive index different from an adjacent portion of the ophthalmic lens 10 that surrounds the sub-structure 14 and is not part of any of the subsurface optical structures 12.

In many embodiments, a refractive index change is defined for each sub-volume of the ophthalmic lens 10 that form the subsurface optical structures 12 so that the resulting subsurface optical structures 12 would provide a desired optical correction when formed within the ophthalmic lens 10. The defined refractive index changes are then used to determine parameters (e.g., laser pulse power (mW), laser pulse width (fs)) of laser pulses that are focused onto the respective sub-volumes to induce the desired refractive index changes in the sub-volumes of the ophthalmic lens 10.

While the sub-structures 14 of the subsurface optical structures 12 have a circular shape in the illustrated embodiment, the sub-structures 14 can have any suitable shape and distribution of refractive index variations. For example, a single sub-structure 14 having an overlapping spiral shape can be employed. In general, one or more substructures 14 having any suitable shapes can be distributed with intervening spaces so as to provide a desired diffraction of light incident on the subsurface optical structure 12ss. More information about subsurface optical structures and forming such structures may be found in U.S. Provisional Application No. 63/001,993, which is incorporated herein by reference in its entirety for all purposes.

In some embodiments, a system including one or more processors may be configured to determine parameters for forming one or more optical structures (e.g., subsurface optical structures) for improving or correcting vision. In some embodiments, the one or more processors of the system may be configured to access a first optical prescription for the patient. The first optical prescription may be prescribed by, for example, an optometrist. The first optical prescription may include one or more prescription parameters for refracting light directed at a retina of the patient so as to improve vision. The prescription parameters may be determined based on any suitable means of measurement. The prescription parameters may specify any suitable parameters for correcting or improving vision. For example, the prescription parameters may include diopter values of sphere, cylinder, or axis. The prescription parameters may include parameters for correcting one or more of a variety of low-order aberrations (e.g., myopia, hyperopia, astigmatism) and high-order aberrations (e.g., spherical aberration, coma, trefoil).

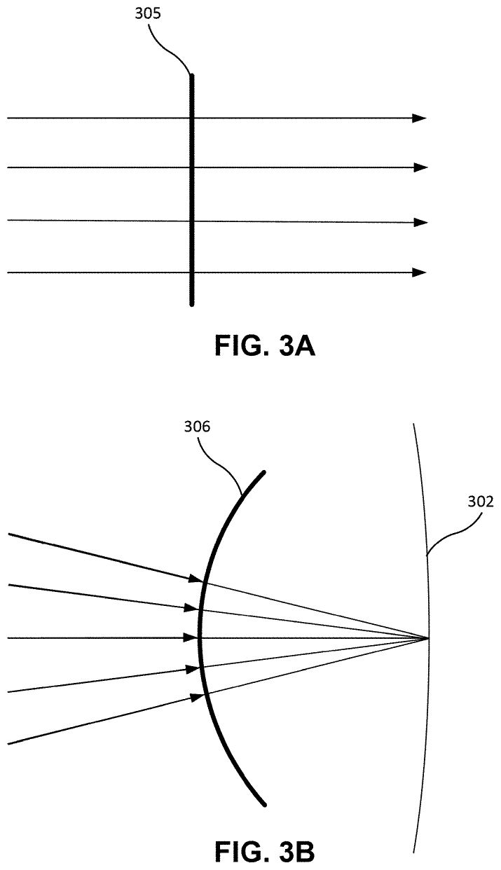

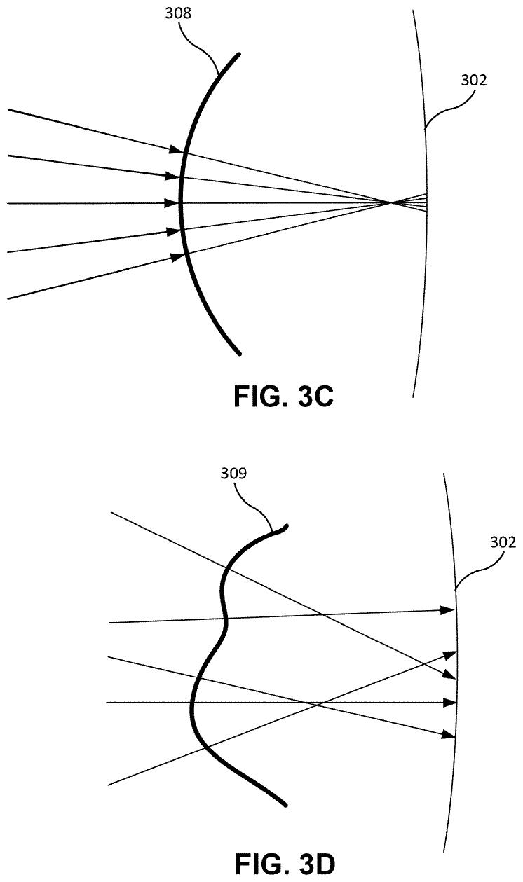

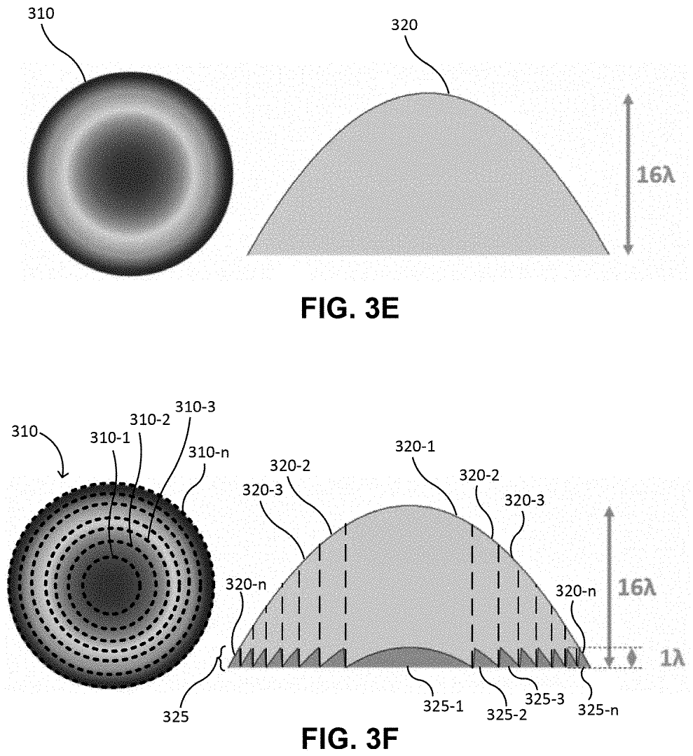

FIGS. 3A-3B illustrate example wavefronts 305, 306 through a medium for parallel and converging rays of light. Prescriptions for correcting or improving vision of a patient can essentially be described as a prescription for creating an optical structure that effects a wavefront configured to modify incoming rays of light before they reach the retina of the patient. A wavefront is an imaginary surface of constant phase. A wavefront can also be thought of as a surface that is normal or perpendicular to rays of light passing through the wavefront. FIG. 3A illustrates a planar wavefront 305 from parallel rays of light. As is evident, the wavefront 305 is perpendicular to the parallel rays of light at each point of intersection. FIG. 3B illustrates a spherical wavefront 306 from converging rays of light. FIG. 3B simulates an ideal configuration of an eye, where the rays of light converge at a single point (on the retina 302). Each of the rays is perpendicular to the wavefront 307 at its respective point of intersection with the wavefront 307. The illustrated rays converge at a single point.

FIGS. 3C-3D illustrate example wavefronts 308, 309 that may simulate aberrations of the eye. Unlike the rays in FIG. 3B, the rays in FIG. 3C do not converge at a single point on the retina 302 (e.g., at or near the macula). Such non-convergence may cause issues with vision by not allowing for a focused image (e.g., causing myopia). FIG. 3D illustrates an aberrated wavefront 309 simulating another aberration of the eye. Again, each of the rays is perpendicular to the wavefront 309 at its respective point of intersection with the wavefront 309. And again, as illustrated, the rays in FIG. 3D do not converge at a single point on the retina 302 (and in fact do not converge at all), causing issues with vision. An appropriate optical structure with a corrective wavefront may be used to correct issues produced by aberrations by, for example, refracting light such that the light rays are made to converge at a single appropriate point on the retina 302. Disclosed herein are methods, devices, and systems for use in forming such optical structures. Although the disclosure focuses on methods, devices, and systems for correcting aberrations of the eye, the disclosure also contemplates enhancing what may be considered normal vision by similar methods, devices, and systems.

FIG. 3E illustrates a two-dimensional wavefront map 310 and a corresponding first variable wavefront 320. In some embodiments, the one or more processors may use the first optical prescription to determine a wavefront for an optical structure for correcting or improving vision of the patient. In some embodiments, the one or more processors may generate a wavefront map, which may be visualized, for example, by the two-dimensional wavefront map 310. The contours of the two-dimensional wavefront map 310 may specify different optical phases of the corresponding wavefront. For example, the different shades in the two-dimensional wavefront map 310 specifies different optical phases of the corresponding wavefront. In some embodiments, the one or more processors may do so by first computing the Zernike coefficient for defocus (C_{2,0}) using the following equation:

C_{2,0}=P*rₘₐₓ²/(4*sqrt(3)), (1)

where P is an add power specified in the first prescription, and rₘₐₓ is the maximum radius of an optical zone.

The Zernike coefficient is a scalar that may be expressed in units of micrometers. In some embodiments, the two-dimensional wavefront map may then be calculated using the following equation:

Wᵤₘ=C_{2,0}*sqrt(3)*(2*ρ²−1), (3)

where ρ is a normalized radial pupil coordinate (radial coordinate/rₘₐₓ)
Wᵤₘ provides a value (e.g., in units of micrometers) for each point of a two-dimensional wavefront map. Referencing FIG. 3D, the two-dimensional wavefront map 310 for a particular optical prescription may be generated using this equation.

In some embodiments, the one or more processors may be configured to generate a first variable wavefront based on the first optical prescription. Referencing FIG. 3D, for example, the first variable wavefront 320 may be generated based on specifications provided by the first optical prescription. The first variable wavefront describes a wavefront in units of waves with respect to a specified wavelength. In some embodiments, the first variable wavefront comprises at least one portion that has a phase height greater than 1.0 wave. In some embodiments, the first variable wavefront may be generated based on the two-dimensional wavefront map. The first variable wavefront may be determined with respect to any desired wavelength by dividing Wᵤₘ for each point by the desired wavelength. For example, the first variable wavefront may be determined with respect to a center of the visible spectrum (e.g., 0.555 μm in daylight). In this example, the equation below may be used to generate a first variable wavefront at 0.555 μm).

W_{WV}=Wᵤₘ/0.555 μm (3)

FIG. 3F illustrates a first phase-wrapped wavefront 325 corresponding to the first variable wavefront 320. In some embodiments, the one or more processors may be configured to phase wrap the first variable wavefront, which may include collapsing the first variable wavefront to generate a first phase-wrapped wavefront. Phase wrapping the first variable wavefront may involve collapsing the first variable wavefront into a wavefront having a predetermined phase height (i.e., the height from peak to valley of the wavefront). For example, referencing FIG. 3B, the first phase-wrapped wavefront 325 may have a phase height of 1.0 wave. Phase-wrapping a variable wavefront to 1.0 wave causes no appreciable change in diffraction or refraction of light rays, and may thus be suitable, for example, for a patient having only myopia. An example Matlab algorithm for phase-wrapping to a phase height of 1.0 wave is shown below, where W555=W_{WV} and Wrap=1:

```
  while cnt == 0
  W555( W555 <-Wrap ) = W555( W555 <-Wrap ) + Wrap;
  if sum( W555(:) <-Wrap ) == 0
    cnt = 1;
  end
end
cnt = 0;
while cnt == 0
  W555( W555 >Wrap) = W555( W555 >Wrap ) - Wrap;
  if sum( W555(:) >Wrap ) == 0
    cnt = 1;
  end
end
```

In some embodiments, collapsing the first variable wavefront may include identifying a plurality of discrete segments of the first variable wavefront. In some embodiments, as is the case in FIG. 3F, each of these discrete segments (e.g., 320-1 to 320-n) may be circumferential discrete segments that extend radially around the two-dimensional wavefront map 310 of the ophthalmic lens. For example, the discrete segment 320-1 in the first variable wavefront 320 may correspond to the portion 310-1 in the two-dimensional wavefront 310, the discrete segment 320-2 may correspond to the segment 310-2, the discrete segment 320-3 may correspond to the segment 310-3, and so on. In other embodiments, the discrete segments may not be circumferential, and the first variable wavefront may be segmented based on, for example, phase height. In the example illustrated in FIG. 3F, each of the discrete segments (325-1 to 325-n) is circumferential, and each discrete segment is adjacent to and concentric with another discrete segment. For example, the discrete segment 325-2 is adjacent to and concentric with the discrete segment 325-1 (similarly, the discrete segment 325-3 is adjacent to and concentric with the discrete segment 325-2, and so on). In some embodiments, the one or more processors of the system may reduce a phase height of each discrete segment by a respective scalar such that a peak of the first discrete segment is at a desired phase height. For example, in FIG. 3F, the phase height of each discrete segment is reduced to a predetermined phase height of 1.0 wave, yielding the first phase-wrapped wavefront 325. As mentioned above, collapsing the first variable wavefront 320 to the phase-wrapped wavefront 325 (which is collapsed to 1.0 wave) causes no appreciable change in diffraction or refraction, and light rays passing an optical structure based on the collapsed phase-wrapped wavefront 325 essentially behave in the same manner as light rays passing an optical structure formed based on the first variable wavefront 320. The resulting phase-wrapped wavefront may include a central discrete segment (e.g., the discrete segment 325-1) and a number of surrounding circumferential, adjacent echelettes (e.g., the discrete segments 325-2 to 325-n) as illustrated in FIG. 3E.

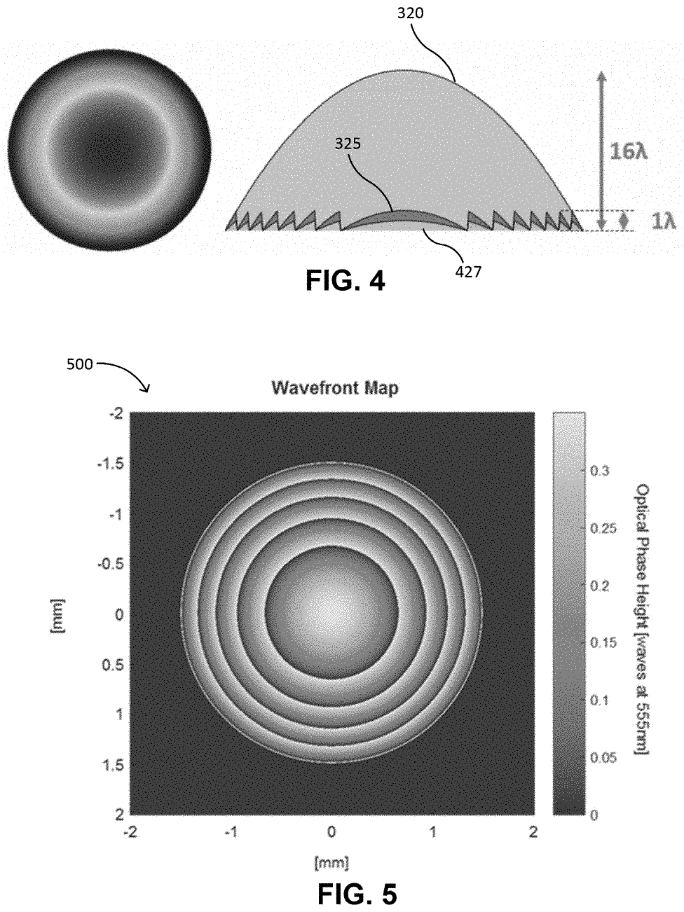

FIG. 4 illustrates a second phase-wrapped wavefront 427 having a phase height less than 1.0 wave. In some embodiments, the system may be configured to phase wrap the first variable wavefront at a predetermined phase height that is not at 1.0 wave to generate a second phase-wrapped wavefront. For example, referencing FIG. 5, the predetermined phase height of the illustrated phase-wrapped wavefront 427 is less than 1.0 wave. As discussed further below, collapsing a wavefront to a phase height other than 1.0 wave causes diffraction, which may be useful for creating a multifocal optical structure. Thus, such a wavefront may be referred to herein as a “diffractive phase-wrapped wavefront.” In some embodiments, the phase-wrapped wavefront may be collapsed at a phase height greater than 1.0 wave. The decision as to whether a wavefront is collapsed to a phase height greater than 1.0 wave or to a phase height less than 1.0 wave may have some practical effects. For example, phase wrapping at greater than 1.0 wave may reduce diffractive chromatic effects. However, phase wrapping to greater than 1.0 wave requires more available refractive index change as compared to phase wrapping to less than 1.0 wave, and any material used is subject to a given range of possible refractive index changes, which may be a limiting factor (e.g., limited by the properties of the material). This may be ultimately overcome in many cases by writing multiple layers or volume filling, however, but there are still limits. So there is a tradeoff between phase wrapping at greater than 1.0 wave or less than 1.0 wave. Whether a wave front is phase-wrapped to less than 1.0 wave or greater than 1.0 wave may also have implications for energy distribution of far/near vision (e.g., for patients with presbyopia), and the practitioner can control this as necessary to achieve a desired effect.

FIG. 5 illustrates a two-dimensional map representation of a phase-wrapped wavefront 500 phase-wrapped at an optical phase height less than 1.0 wave, such as the wavefront 427 in

FIG. 4. The illustrated phase-wrapped wavefront has a 3.0 mm diameter optical zone and a diffractive bifocal with 2.5 Diopters (D) of add-power. The diffractive bifocal wavefront is designed to have an optical phase height of 0.35 waves at 555 nm wavelength. As illustrated, the phase-wrapped wavefront 500 includes five discrete circumferential segments, each segment gradually decreasing in phase height (from 0.35 waves to 0 waves) from an inner boundary of the segment to an outer boundary of the segment.

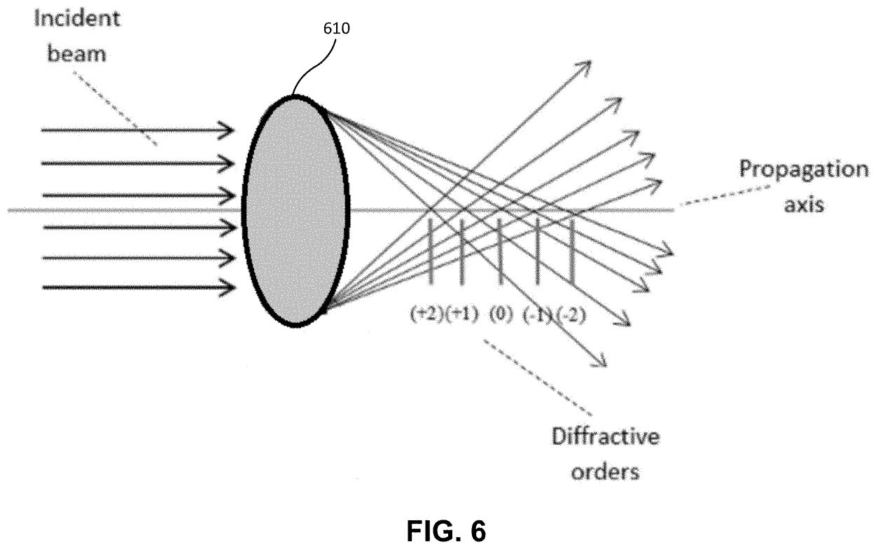

FIG. 6 illustrates an example of an optical structure 610 having diffractive properties. In some embodiments, an optical structure having a phase-wrapped wavefront collapsed at a phase height other than 1.0 wave (e.g., less than 1.0 wave) has diffractive effects that create multiple focal points, which may be useful, for example, in correcting vision in patients having presbyopia. As illustrated in FIG. 6, light rays passing through the optical structure 610, which is an optical structure with diffractive properties, an incident beam can be focused simultaneously at several positions along the propagation axis. Diffraction in this manner can be used to create multiple focal points, for example, to improve the vision of patients with presbyopia. For example, an optical structure having diffractive properties may have a first focal point for near-vision and a second focal point for far-vision.

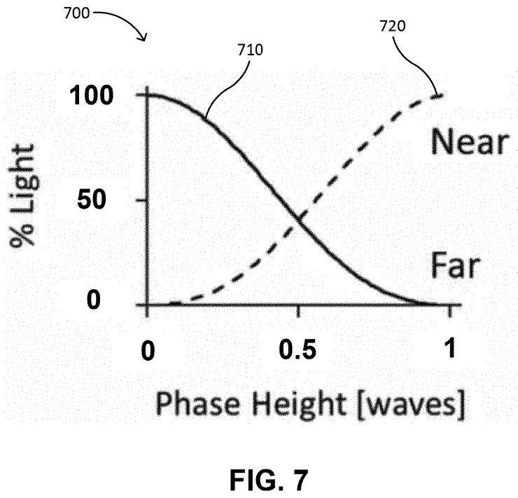

FIG. 7 is a graph 700 illustrating the relative distribution of light between a near-vision focal point and a far-vision focal point as phase height of a wavefront is adjusted between 0 wave and 1.0 wave. In some embodiments, the system may generate diffractive phase-wrapped wavefronts (e.g., phase-wrapped wavefronts at less than 1.0 wave or greater than 1.0 wave), for conditions such as presbyopia that are designed to provide both high optical quality for far-vision and intermediate- and near-vision (e.g., good through-focus image quality), but with the understanding that there may be a trade-off. An example representation of this trade-off is illustrated in FIG. 7. As illustrated by the far-vision curve 710, as the phase height increases to 1.0 wave, the percentage of light distributed to the far-vision focal point by the diffraction of incoming light decreases (and therefore image quality for far-vision generally decreases). By contrast, referencing the near-vision curve 720, as the phase height increases to 1.0 wave, the percentage of light distributed to the near-vision focal point increases (and therefore image quality for near-vision generally increases). In some embodiments, a desired distribution for this tradeoff may be specified in an optical prescription (e.g., as add power), and may be determined based on any suitable of patient-dependent factors. For example, the patient who often engages in high-detail work (e.g., a watchmaker) may require a relatively high add power (e.g., 4.0 diopters). A relatively low add power (e.g., 1.0 diopters) may be suitable for a patient who does not engage in such high-detail work. A diffractive phase-wrapped wavefront may be generated with a prescription having such considerations in mind to come to a desired trade-off.

In some embodiments, the one or more processors may be configured to generate multiple wavefronts, for example, to correct multiple aberrations of the eye. In some embodiments, the one or more processors may generate a second variable wavefront based on a second optical prescription, wherein the second optical prescription comprises an add power for multifocal vision correction. The term second optical prescription does not necessarily reference a separate prescription, and may instead refer to separate one or more parameters for correcting a different aberration than the first optical prescription. For example, a patient may receive a single prescription from an optometrist for correcting near-vision based on parameters of a first optical prescription and for correcting far-vision based on parameters of a second optical prescription (e.g., including an add power). In some embodiments, the one or processors may phase-wrap the second variable wavefront, wherein phase wrapping the second variable wavefront comprises collapsing the second variable wavefront to a second phase-wrapped wavefront having a second predetermined phase height. The second predetermined phase height may be less than 1.0 wave, so as to allow for diffractive effects as discussed above. In some embodiments, a first phase-wrapped wavefront may have a phase height of 1.0 wave, and the second phase-wrapped wavefront may have phase height less than 1.0 wave. In these embodiments, the first phase-wrapped wavefront may be useful for correcting myopia and the second phase-wrapped wavefront may be useful for correcting presbyopia, for example.

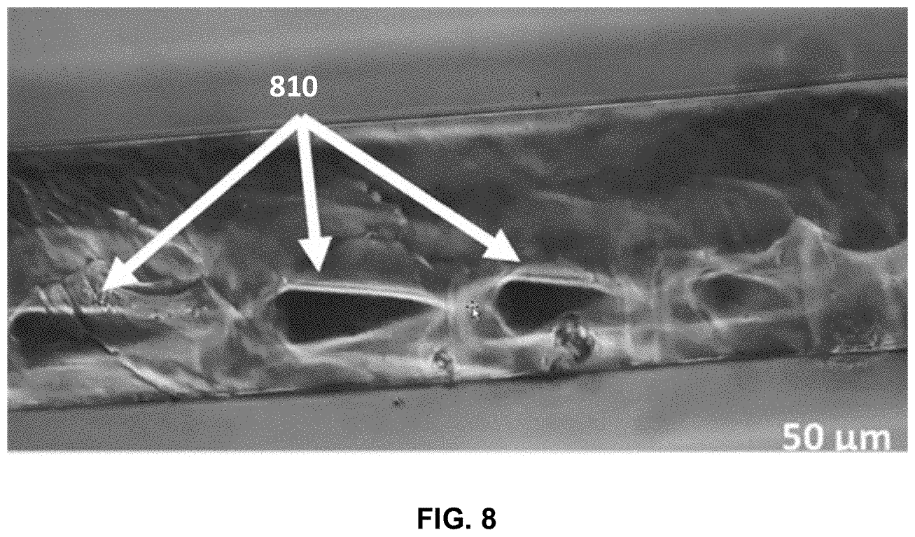

FIG. 8 illustrates a cross section of an ophthalmic lens including a subsurface optical structure having multiple substructures 810. In some embodiments, the one or more processors may be configured to generate, based on the first phase-wrapped wavefront, energy output parameters for forming a first optical structure using an energy source. In some embodiments, the first optical structure may be configured to refract light directed at the retina of the patient so as to improve vision. In some embodiments, the optical structure may be a subsurface optical structure. For example, referencing the cross-section illustrated in FIG. 8, the optical structure may be a subsurface optical structure having multiple substructures 810 that may be concentric. As discussed in further detail above, subsurface optical structures may be achieved by focusing laser pulses appropriately to depths within the ophthalmic lens such that changes in refractive property occur to sub-volumes in the interior of the ophthalmic lens.

The conventional approach for forming a diffractive ophthalmic lens involves creating Fresnel rings that project outward from the exterior of the ophthalmic lens. Such a configuration not only increases the thickness profile of the lens, but it may also cause issues with the optical properties of the ophthalmic lens. For example, in the case of a contact lens, disposing Fresnel rings on the outward-facing side of the contact lens may cause errors in light diffraction or refraction because the level of tear film may vary across the peaks and valleys of the Fresnel rings. And disposing the Fresnel rings on the inward-facing side of the contact lens may cause patient discomfort.

Moreover, conventional approaches rely on changes in the thickness of ophthalmic lenses to supply the base power of the ophthalmic lenses. In these approaches, the refractive index of the material throughout an ophthalmic lens may remain constant. This reliance on thickness necessarily means that lenses with relatively high base powers are relatively thick. For contact lenses, this may mean patient discomfort. For IOLs, this may mean an increase in patient risk during surgery, and a higher potential for complications (e.g., because it may be more difficult to get the IOL seated in the capsular bag). By contrast, the disclosed methods of creating subsurface optical structures using an energy system (e.g., a laser) does not rely on changing the thickness of an ophthalmic lens for the base power. Rather, as explained above, refractive indices of subvolumes within the ophthalmic lens are modified to supply the base power of the ophthalmic lens and thereby refract and/or diffract light as desired. Finally the use of an energy system as described below with respect to optical zones provides increased resolution as compared to more conventional techniques such as cryolathes or molded injection.

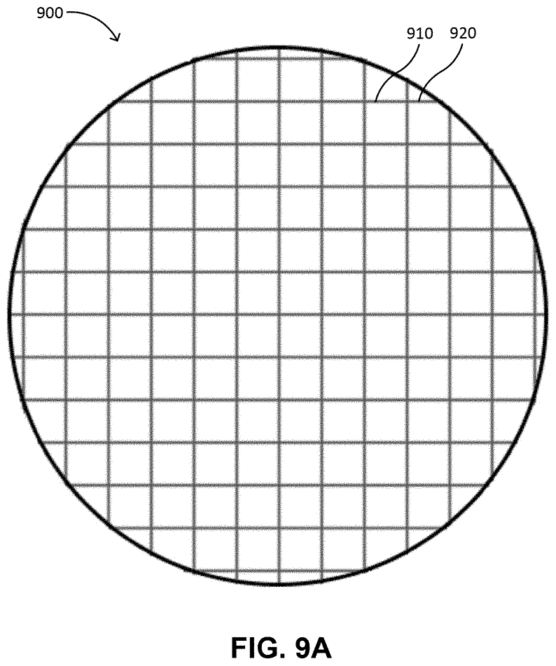

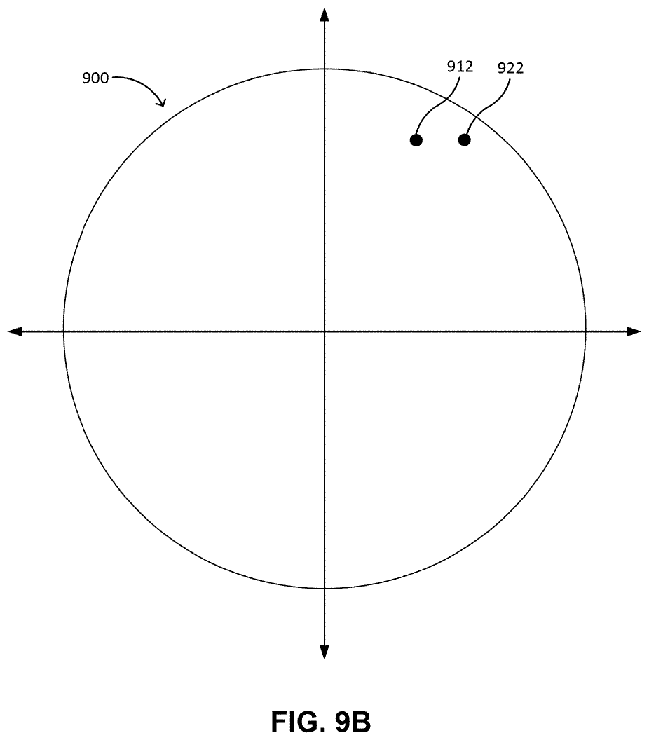

FIGS. 9A-9B illustrate example conceptualizations of an ophthalmic lens 900 having a plurality of optical zones. In some embodiments, an ophthalmic lens may be divided up into a plurality of pixels, each pixel corresponding to an optical zone. An optical zone may be a sub-region or a sub-volume of an ophthalmic lens. This is illustrated in FIG. 9A, which shows the ophthalmic lens 900 divided up into a plurality of pixels (e.g., the pixels 910 and 920) in a grid fashion. Although FIG. 9A illustrates uniform pixels that are square shaped, this disclosure contemplates that pixels may be of any suitable shape (e.g., hexagonal, pentagonal, circular) and that they may not be uniform (e.g., they may of different shapes and sizes). A pixel area may correspond to the resolution of an energy delivery system (e.g., a laser system) configured to form an optical structure corresponding to a phase-wrapped wavefront. That is, a pixel area may correspond to a minimum area of a sub-region of the ophthalmic lens at which the energy delivery system may focus an energy beam (e.g., a laser pulse) to change a refractive index of the sub-volume associated with the sub-region. FIG. 9B illustrates another conceptualization of optical zones, where the ophthalmic lens is not divided up into discrete pixels. Instead, the ophthalmic lens is mapped out using a coordinate system (e.g., a two-dimensional x-y coordinate system, or a three-dimensional x-y-z coordinate system). For example, the points 912 and 922 may each have a respective coordinate in the coordinate system.

In some embodiments, the generated energy output parameters may specify an amount of power that is to be delivered by the energy delivery system at one or more optical zones. For example, referencing FIG. 9A, the energy output parameters may specify power levels (e.g., in Watts) for one or more laser pulses that are to be delivered by a laser system at the pixel 910 and the pixel 920. Similarly, referencing FIG. 9B, the energy output parameters may specify power levels for a plurality of coordinates associated with the ophthalmic lens (e.g., the points 912 and 922). In some embodiments, the generated energy output parameters may specify a duration during which energy beam may be directed at one or more optical zones. For example, the energy output parameters may specify pulse durations for directing a laser beam at one or more of the optical zones. In some embodiments, the energy output parameters may specify a depth at which energy beam is to be delivered in forming an optical structure. For example, the energy output parameters may specify that a first set of pulses is to be delivered to a set of optical zones at a first depth along a first layer of the ophthalmic lens, and may further specify that a second set of pulses is to be delivered to a second set of optical zones at a second depth along a second layer of the ophthalmic lens. In this example, the first layer may be based on a phase-wrapped wavefront collapsed at 1.0 wave (e.g., for correcting myopia), and the second layer may be based on a phase-wrapped wavefront collapsed at less than 1.0 wave (e.g., for correcting presbyopia). The first set of pulses in this example may be associated with a first set of energy output parameters (e.g., power levels, pulse durations, depths) for a plurality of optical zones, and the second set of pulses in this example may be associated with a second set of energy output parameters.

In some embodiments, the one or more processors, and generating the energy output parameters, may apply a calibration function so as to create a tailored set of parameters for real-world conditions. The calibration function may depend on any suitable factors. For example, the one or more processors may apply a calibration function based on one or more of a material property of the ophthalmic lens, a gender of the patient, an age of the patient, a depth at which an optical structure (e.g., a subsurface optical structure) is to be formed in the ophthalmic lens, a number of layers, the distance by which different layers are separated, and/or properties of an energy source for which the energy output parameters are generated (e.g., scan speed, numerical aperture, wavelength, pulse width, repetition rate, writing depth, line-spacing, scan architecture).

In some embodiments, the one or more processors may be configured to generate energy output parameters for forming multiple optical structures. For example, the one or more processors may generate energy output parameters for forming a first subsurface optical structure based on a first phase-wrapped wavefront having a phase height of 1.0 wave (e.g., for correcting myopia) and a second subsurface optical structure based on a second phase-wrapped wavefront having a phase height less than 1.0 wave so as to diffract light (e.g., for correcting presbyopia). In these embodiments, what results may be a multifocal ophthalmic lens configured to create multiple focal points within the eye. In some embodiments, these optical structures may be formed as distinct layers (e.g., in a cornea, a contact lens, an intraocular lens). In other embodiments, the one or more processors may generate parameters for forming a single optical structure as a single layer that combines the first phase-wrapped wavefront and the second phase-wrapped wavefront such that the single layer has the effects specified by the two wavefronts.

In some embodiments, the system may further include an energy source configured to direct one or more energy beams toward the optical structure so as to form the first optical structure based on the energy output parameters. In other embodiments, the system may not include such an energy source, and may simply send the energy output parameters to a different system that includes an energy source for forming optical structures. In some embodiments, the energy source may be a laser source configured to deliver targeted pulsed or continuous-wave laser beams.

Although the examples in the disclosure focus on correction of standard sphere/cylinder error and/or presbyopia, the disclosure contemplates the generation of wavefronts that may be used to form optical structures for correcting any suitable aberration (e.g., customized higher order aberrations, myopia progression peripheral error). For example, wavefronts described by any combination of Zernike polynomials may be generated. Although the disclosure focus is on subsurface optical structures, disclosure contemplates any suitable optical structures, for example, optical structures that are not subsurface.

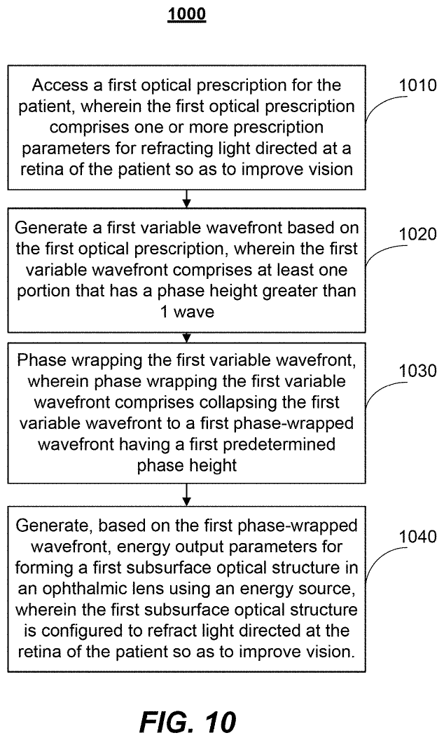

FIG. 10 illustrates an example method 1000 for determining parameters for forming a subsurface optical structure for improving vision in a patient. The method may include, at step 1010, accessing a first optical prescription for the patient, wherein the first optical prescription comprises one or more prescription parameters for refracting light directed at a retina of the patient so as to improve vision. At step 1020, the method may include generating a first variable wavefront based on the first optical prescription, wherein the first variable wavefront comprises at least one portion that has a phase height greater than 1.0 wave. At step 1030, the method may include phase wrapping the first variable wavefront, wherein phase wrapping the first variable wavefront comprises collapsing the first variable wavefront to a first phase-wrapped wavefront having a first predetermined phase height. At step 1040, the method may include generating, based on the first phase-wrapped wavefront, energy output parameters for forming a first subsurface optical structure in an ophthalmic lens using an energy source, wherein the first subsurface optical structure is configured to refract light directed at the retina of the patient so as to improve vision.

Particular embodiments may repeat one or more steps of the method of FIG. 10, where appropriate. Although this disclosure describes and illustrates particular steps of the method of FIG. 10 as occurring in a particular order, this disclosure contemplates any suitable steps of the method of FIG. 10 occurring in any suitable order. Moreover, although this disclosure describes and illustrates an example method for determining parameters for forming a subsurface optical structure for improving vision in a patient, including the particular steps of the method of FIG. 10, this disclosure contemplates any suitable method for determining parameters for forming a subsurface optical structure for improving vision in a patient, including any suitable steps, which may include all, some, or none of the steps of the method of FIG. 10, where appropriate.

Furthermore, although this disclosure describes and illustrates particular components, devices, or systems carrying out particular steps of the method of FIG. 10, this disclosure contemplates any suitable combination of any suitable components, devices, or systems carrying out any suitable steps of the method of FIG. 10.

Example 1 is a method of determining parameters for forming a subsurface optical structure in an ophthalmic lens for improving vision in a patient. The example 1 method includes: (1) accessing a first optical prescription for the patient, wherein the first optical prescription comprises one or more prescription parameters for refracting light directed at a retina of the patient so as to improve vision; (2) generating a first variable wavefront based on the first optical prescription, wherein the first variable wavefront comprises at least one portion that has a phase height greater than 1.0 wave; (3) phase wrapping the first variable wavefront, wherein phase wrapping the first variable wavefront comprises collapsing the first variable wavefront to a first phase-wrapped wavefront having a first predetermined phase height; and (4) generating, based on the first phase-wrapped wavefront, energy output parameters for forming a first subsurface optical structure in the ophthalmic lens using an energy source, wherein the first subsurface optical structure is configured to refract light directed at the retina of the patient so as to improve vision.

Example 2 is the method of example 1 (or of any other preceding or subsequent examples individually or in combination), wherein the one or more prescription parameters comprise diopter values of sphere, cylinder, or axis.

Example 3 is the method of example 1 (or of any other preceding or subsequent examples individually or in combination), wherein collapsing the first variable wavefront includes: (1) identifying a first discrete segment of the first variable wavefront; (2) reducing a phase height of the first discrete segment by a first scalar such that a peak of the first discrete segment is at the first predetermined phase height; (3) identifying a second discrete segment of the first variable wavefront, wherein the second discrete segment is substantially concentric with the first discrete segment; and (4) reducing a phase height of the second discrete segment by a second scalar such that a peak of the second discrete segment is at the first predetermined phase height.

Example 4 is the method of example 3 (or of any other preceding or subsequent examples individually or in combination), wherein the first subsurface optical structure is configured to improve presbyopia, and wherein the first predetermined phase height is not equal to an integer number of waves.

Example 5 is the method of example 3 (or of any other preceding or subsequent examples individually or in combination), wherein the first subsurface optical structure is configured to improve myopia, and wherein the first predetermined phase height is an integer number of waves for a phase-wrapped wavefront.

Example 6 is the method of example 1 (or of any other preceding or subsequent examples individually or in combination), wherein the first predetermined phase height is 1.0 wave, the method further includes: (1) generating a second variable wavefront based on a second optical prescription, wherein the second optical prescription comprises an add power for multifocal vision correction; and (2) phase wrapping the second variable wavefront, wherein phase wrapping the second variable wavefront comprises collapsing the second variable wavefront to a second phase-wrapped wavefront having a second predetermined phase height, wherein the second predetermined phase height is less than 1.0 wave.

Example 7 is the method of example 6 (or of any other preceding or subsequent examples individually or in combination), further including generating, based on the second phase-wrapped wavefront, energy output parameters for forming a second subsurface optical structure in an optical structure using an energy source, wherein the second subsurface optical structure is configured to diffract light so as to create multiple focal points.

Example 8 is the method of example 7 (or of any other preceding or subsequent examples individually or in combination), wherein the first subsurface optical structure is configured to improve low order aberrations and the second subsurface optical structure is configured to improve presbyopia, the first subsurface optical structure and the second subsurface optical structure in combination forming a multifocal refractive structure.

Example 9 is the method of example 6 (or of any other preceding or subsequent examples individually or in combination), wherein the energy output parameters for forming the first subsurface optical structure are further based on the second phase-wrapped wavefront such that the first subsurface optical structure is configured to be a single multifocal subsurface optical structure.

Example 10 is the method of any one of example 1 through example 9 (or of any other preceding or subsequent examples individually or in combination), wherein the energy output parameters specify a plurality of power levels corresponding to a plurality of optical zones on the ophthalmic lens, the method further including: (1) directing a first energy beam from the energy source at a first optical zone on the ophthalmic lens for a first duration, wherein a power level of the first energy beam is based on a corresponding power level as specified by the energy output parameters; and (2) directing a second energy beam from the energy source at a second optical zone on the ophthalmic lens for a second duration, wherein a power level of the second energy beam is based on a corresponding power level as specified by the energy output parameters; wherein the first energy beam and the second energy beam alter refractive indexes of the first optical zone and the second optical zone, respectively, and wherein the first subsurface optical structure comprises the first optical zone and the second optical zone.

Example 11 is the method of example 10 (or of any other preceding or subsequent examples individually or in combination), wherein the first subsurface optical structure is formed within an interior of the ophthalmic lens.

Example 12 is the method of any one of example 1 through example 9 (or of any other preceding or subsequent examples individually or in combination), wherein the first variable wavefront comprises a two-dimensional wavefront.

Example 13 is the method of any one of example 1 through example 9 (or of any other preceding or subsequent examples individually or in combination), wherein the energy source comprises a laser.

Example 14 is the method of any one of example 1 through example 9 (or of any other preceding or subsequent examples individually or in combination), wherein the ophthalmic lens is an intraocular lens, a contact lens, or a cornea of the patient.

Example 15 is the method of any one of example 1 through example 9 (or of any other preceding or subsequent examples individually or in combination), wherein generating the energy output parameters comprises applying a calibration function based on a material property of the ophthalmic lens, a gender of the patient, or an age of the patient.

Example 16 is the method of any one of example 1 through example 9 (or of any other preceding or subsequent examples individually or in combination), wherein generating the energy output parameters comprises applying a calibration function based on a depth at which the first subsurface optical structure is to be formed in the ophthalmic lens.

Example 17 is a system for forming one or more subsurface optical structures in an ophthalmic lens for improving vision in a patient. The system of example 17 includes one or more processors configured to: (1) access a first optical prescription for the patient, wherein the first optical prescription comprises one or more prescription parameters for refracting light directed at a retina of the patient so as to improve vision; (2) generate a first variable wavefront based on the first optical prescription, wherein the first variable wavefront comprises at least one portion that has a phase height greater than 1.0 wave; (3) phase wrap the first variable wavefront, wherein phase wrapping the first variable wavefront comprises collapsing the first variable wavefront to a first phase-wrapped wavefront has a first predetermined phase height; and (4) generate, based on the first phase-wrapped wavefront, energy output parameters for forming a first subsurface optical structure in the ophthalmic lens using an energy source, wherein the first subsurface optical structure is configured to refract light directed at the retina of the patient so as to improve vision. The system of example 17 further includes an energy source configured to direct one or more energy beams toward the ophthalmic lens so as to form the first subsurface optical structure in the ophthalmic lens based on the energy output parameters.

Example 18 is the system of example 17 (or of any other preceding or subsequent examples individually or in combination), wherein the one or more prescription parameters comprise diopter values of sphere, cylinder, or axis.

Example 19 is the system of example 17 (or of any other preceding or subsequent examples individually or in combination), wherein the one or more processors are configured to collapse the first variable wavefront at least in part by: (1) identifying a first discrete segment of the first variable wavefront; (2) reducing a phase height of the first discrete segment by a first scalar such that a peak of the first discrete segment is at the first predetermined phase height; (3) identifying a second discrete segment of the first variable wavefront, wherein the second discrete segment is substantially concentric with the first discrete segment; and (4) reducing a phase height of the second discrete segment by a second scalar such that a peak of the second discrete segment is at the first predetermined phase height.

Example 20 is the system of example 19 (or of any other preceding or subsequent examples individually or in combination), wherein the first subsurface optical structure is configured to improve presbyopia, and wherein the first predetermined phase height is less than 1.0 wave.

Example 21 is the system of example 19 (or of any other preceding or subsequent examples individually or in combination), wherein the first subsurface optical structure is configured to improve myopia, and wherein the first predetermined phase height is 1.0 wave.

Example 22 is the system of example 17 (or of any other preceding or subsequent examples individually or in combination), wherein the first predetermined phase height is 1.0 wave, and wherein the one or more processors are further configured to: (1) generate a second variable wavefront based on a second optical prescription, wherein the second optical prescription comprises an add power for multifocal vision correction; and (2) phase wrap the second variable wavefront, wherein phase wrapping the second variable wavefront comprises collapsing the second variable wavefront to a second phase-wrapped wavefront having a second predetermined phase height, wherein the second predetermined phase height is less than 1.0 wave.

Example 23 is the system of example 22 (or of any other preceding or subsequent examples individually or in combination), wherein the one or more processors are further configured to generate, based on the second phase-wrapped wavefront, energy output parameters for forming a second subsurface optical structure in an optical structure using an energy source, wherein the second subsurface optical structure is configured to diffract light so as to create multiple focal points.

Example 24 is the system of example 23 (or of any other preceding or subsequent examples individually or in combination), wherein the first subsurface optical structure is configured to improve myopia and the second subsurface optical structure is configured to improve presbyopia, the first subsurface optical structure and the second subsurface optical structure in combination forming a multifocal refractive structure.

Example 25 is the system of example 22 (or of any other preceding or subsequent examples individually or in combination), wherein the energy output parameters for forming the first subsurface optical structure are further based on the second phase-wrapped wavefront such that the first subsurface optical structure is configured to be a single multifocal subsurface optical structure.

Example 26 is the system of any one of example 17 through example 25 (or of any other preceding or subsequent examples individually or in combination), wherein the energy output parameters specify a plurality of power levels corresponding to a plurality of optical zones on the ophthalmic lens, and wherein the energy source is configured to: (1) direct a first energy beam from the energy source at a first optical zone on the ophthalmic lens for a first duration, wherein a power level of the first energy beam is based on a corresponding power level as specified by the energy output parameters; and (2) direct a second energy beam from the energy source at a second optical zone on the ophthalmic lens for a second duration, wherein a power level of the second energy beam is based on a corresponding power level as specified by the energy output parameters; wherein the first energy beam and the second energy beam alter refractive indexes of the first optical zone and the second optical zone, respectively, and wherein the first subsurface optical structure comprises the first optical zone and the second optical zone.

Example 27 is the system of example 26 (or of any other preceding or subsequent examples individually or in combination), wherein the first subsurface optical structure is formed within an interior of the ophthalmic lens.

Example 28 is the system of any one of example 17 through example 25 (or of any other preceding or subsequent examples individually or in combination), wherein the first variable wavefront comprises a two-dimensional wavefront.

Example 29 is the system of any one of example 17 through example 25 (or of any other preceding or subsequent examples individually or in combination), wherein the energy source comprises a laser.

Example 30 is the system of any one of example 17 through example 25 (or of any other preceding or subsequent examples individually or in combination), wherein the ophthalmic lens is an intraocular lens, a contact lens, or a cornea of the patient.

Example 31 is the system of any one of example 17 through example 25 (or of any other preceding or subsequent examples individually or in combination), wherein the one or more processors are configured to generate the energy output parameters by at least applying a calibration function based on a material property of the ophthalmic lens, a gender of the patient, or an age of the patient.

Example 32 is the system of any one of example 17 through example 25 (or of any other preceding or subsequent examples individually or in combination), wherein the one or more processors are configured to generate the energy output parameters by at least applying a calibration function based on a depth at which the first subsurface optical structure is to be formed in the ophthalmic lens.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A method of determining parameters for forming a subsurface optical structure in an ophthalmic lens for improving vision in a patient, the method comprising:
accessing a first optical prescription for the patient, wherein the first optical prescription comprises one or more prescription parameters for refracting light directed at a retina of the patient so as to improve vision;
generating a first variable wavefront based on the first optical prescription, wherein the first variable wavefront comprises at least one portion that has a phase height greater than 1.0 wave;
phase wrapping the first variable wavefront, wherein phase wrapping the first variable wavefront comprises collapsing the first variable wavefront to a first phase-wrapped wavefront having a first predetermined phase height; and
generating, based on the first phase-wrapped wavefront, energy output parameters for forming a first subsurface optical structure in the ophthalmic lens using an energy source, wherein the first subsurface optical structure is configured to refract light directed at the retina of the patient so as to improve vision.

2. The method of claim 1, wherein the one or more prescription parameters comprise diopter values of sphere, cylinder, or axis.

3. The method of claim 1, wherein collapsing the first variable wavefront comprises:
identifying a first discrete segment of the first variable wavefront;
reducing a phase height of the first discrete segment by a first scalar such that a peak of the first discrete segment is at the first predetermined phase height;
identifying a second discrete segment of the first variable wavefront, wherein the second discrete segment is substantially concentric with the first discrete segment; and
reducing a phase height of the second discrete segment by a second scalar such that a peak of the second discrete segment is at the first predetermined phase height.

4. The method of claim 3, wherein the first subsurface optical structure is configured to improve presbyopia, and wherein the first predetermined phase height is not equal to an integer number of waves.

5. The method of claim 3, wherein the first subsurface optical structure is configured to improve myopia, and wherein the first predetermined phase height is an integer number of waves for a phase-wrapped wavefront.

6. The method of claim 1, wherein the first predetermined phase height is 1.0 wave, the method further comprising:
generating a second variable wavefront based on a second optical prescription, wherein the second optical prescription comprises an add power for multifocal vision correction; and
phase wrapping the second variable wavefront, wherein phase wrapping the second variable wavefront comprises collapsing the second variable wavefront to a second phase-wrapped wavefront having a second predetermined phase height, wherein the second predetermined phase height is less than 1.0 wave.

7. The method of claim 6, further comprising generating, based on the second phase-wrapped wavefront, energy output parameters for forming a second subsurface optical structure in an optical structure using an energy source, wherein the second subsurface optical structure is configured to diffract light so as to create multiple focal points.

8. The method of claim 7, wherein the first subsurface optical structure is configured to improve low order aberrations and the second subsurface optical structure is configured to improve presbyopia, the first subsurface optical structure and the second subsurface optical structure in combination forming a multifocal refractive structure.

9. The method of claim 6, wherein the energy output parameters for forming the first subsurface optical structure are further based on the second phase-wrapped wavefront such that the first subsurface optical structure is configured to be a single multifocal subsurface optical structure.

10. The method of claim 1, wherein the energy output parameters specify a plurality of power levels corresponding to a plurality of optical zones on the ophthalmic lens, the method further comprising:
directing a first energy beam from the energy source at a first optical zone on the ophthalmic lens for a first duration, wherein a power level of the first energy beam is based on a corresponding power level as specified by the energy output parameters; and
directing a second energy beam from the energy source at a second optical zone on the ophthalmic lens for a second duration, wherein a power level of the second energy beam is based on a corresponding power level as specified by the energy output parameters;
wherein the first energy beam and the second energy beam alter refractive indexes of the first optical zone and the second optical zone, respectively, and wherein the first subsurface optical structure comprises the first optical zone and the second optical zone.

11. The method of claim 10, wherein the first subsurface optical structure is formed within an interior of the ophthalmic lens.

12. The method of claim 1, wherein the first variable wavefront comprises a two-dimensional wavefront.

13. The method of claim 1, wherein the energy source comprises a laser.

14. The method of claim 1, wherein the ophthalmic lens is an intraocular lens, a contact lens, or a cornea of the patient.

15. The method of claim 1, wherein generating the energy output parameters comprises applying a calibration function based on a material property of the ophthalmic lens, a gender of the patient, or an age of the patient.

16. The method of claim 1, wherein generating the energy output parameters comprises applying a calibration function based on a depth at which the first subsurface optical structure is to be formed in the ophthalmic lens.

17. A system for forming one or more subsurface optical structures in an ophthalmic lens for improving vision in a patient, the system comprising:
one or more processors configured to:
access a first optical prescription for the patient, wherein the first optical prescription comprises one or more prescription parameters for refracting light directed at a retina of the patient so as to improve vision;
generate a first variable wavefront based on the first optical prescription, wherein the first variable wavefront comprises at least one portion that has a phase height greater than 1.0 wave;
phase wrap the first variable wavefront, wherein phase wrapping the first variable wavefront comprises collapsing the first variable wavefront to a first phase-wrapped wavefront has a first predetermined phase height; and
generate, based on the first phase-wrapped wavefront, energy output parameters for forming a first subsurface optical structure in the ophthalmic lens using an energy source, wherein the first subsurface optical structure is configured to refract light directed at the retina of the patient so as to improve vision; and
an energy source configured to direct one or more energy beams toward the ophthalmic lens so as to form the first subsurface optical structure in the ophthalmic lens based on the energy output parameters.

18. The system of claim 17, wherein the one or more prescription parameters comprise diopter values of sphere, cylinder, or axis.

19. The system of claim 17, wherein the one or more processors are configured to collapse the first variable wavefront at least in part by:
identifying a first discrete segment of the first variable wavefront;
reducing a phase height of the first discrete segment by a first scalar such that a peak of the first discrete segment is at the first predetermined phase height;
identifying a second discrete segment of the first variable wavefront, wherein the second discrete segment is substantially concentric with the first discrete segment; and
reducing a phase height of the second discrete segment by a second scalar such that a peak of the second discrete segment is at the first predetermined phase height.

20. The system of claim 19, wherein the first subsurface optical structure is configured to improve presbyopia, and wherein the first predetermined phase height is less than 1.0 wave.

21. The system of claim 19, wherein the first subsurface optical structure is configured to improve myopia, and wherein the first predetermined phase height is 1.0 wave.

22. The system of claim 17, wherein the first predetermined phase height is 1.0 wave, and wherein the one or more processors are further configured to:
generate a second variable wavefront based on a second optical prescription, wherein the second optical prescription comprises an add power for multifocal vision correction; and
phase wrap the second variable wavefront, wherein phase wrapping the second variable wavefront comprises collapsing the second variable wavefront to a second phase-wrapped wavefront having a second predetermined phase height, wherein the second predetermined phase height is less than 1.0 wave.

23. The system of claim 22, wherein the one or more processors are further configured to generate, based on the second phase-wrapped wavefront, energy output parameters for forming a second subsurface optical structure in an optical structure using an energy source, wherein the second subsurface optical structure is configured to diffract light so as to create multiple focal points.

24. The system of claim 23, wherein the first subsurface optical structure is configured to improve myopia and the second subsurface optical structure is configured to improve presbyopia, the first subsurface optical structure and the second subsurface optical structure in combination forming a multifocal refractive structure.

25. The system of claim 22, wherein the energy output parameters for forming the first subsurface optical structure are further based on the second phase-wrapped wavefront such that the first subsurface optical structure is configured to be a single multifocal subsurface optical structure.

26. The system of claim 17, wherein the energy output parameters specify a plurality of power levels corresponding to a plurality of optical zones on the ophthalmic lens, and wherein the energy source is configured to:
direct a first energy beam from the energy source at a first optical zone on the ophthalmic lens for a first duration, wherein a power level of the first energy beam is based on a corresponding power level as specified by the energy output parameters; and
direct a second energy beam from the energy source at a second optical zone on the ophthalmic lens for a second duration, wherein a power level of the second energy beam is based on a corresponding power level as specified by the energy output parameters;
wherein the first energy beam and the second energy beam alter refractive indexes of the first optical zone and the second optical zone, respectively, and wherein the first subsurface optical structure comprises the first optical zone and the second optical zone.

27. The system of claim 26, wherein the first subsurface optical structure is formed within an interior of the ophthalmic lens.

28. The system of claim 17, wherein the first variable wavefront comprises a two-dimensional wavefront.

29. The system of claim 17, wherein the energy source comprises a laser.

30. The system of claim 17, wherein the ophthalmic lens is an intraocular lens, a contact lens, or a cornea of the patient.

31. The system of claim 17, wherein the one or more processors are configured to generate the energy output parameters by at least applying a calibration function based on a material property of the ophthalmic lens, a gender of the patient, or an age of the patient.

32. The system of claim 17, wherein the one or more processors are configured to generate the energy output parameters by at least applying a calibration function based on a depth at which the first subsurface optical structure is to be formed in the ophthalmic lens.
